(19) United States
(12) Patent Application Publication
(10) Pub. No.: US 2008/0119923 A1
(43) Pub. Date: May 22, 2008

(54) Bifurcated stent delivery system

(75) Inventors: Thomas Trinh Tran, Coon Rapids, MN (US); Tracee Eidenschink, Wayzata, MN (US); Jan Weber, Maple Grove, MN (US)

Correspondence Address: MERCHANT & GOULD PC, P.O. BOX 2903, MINNEAPOLIS, MN, 55402-0903, US

(73) Assignee: Scimed Life Systems, Inc., Maple Grove, MN (US)

(21) Appl. No.: 12/011,082

(22) Filed: Jan. 23, 2008

Related U.S. Application Data
(63) Continuation of application No. 10/863,724, filed on Jun. 8, 2004.

(51) Int. Cl. A61F 2/06

(52) U.S. Cl. 623/1.11

(57) ABSTRACT

A stent delivery system comprises a catheter which includes a catheter shaft and a balloon positioned thereon. A rotatable sheath is rotatably disposed about a portion of the catheter. The rotatable sheath has a distal portion which extends over the balloon and a proximal portion which extends over the catheter shaft proximal to the balloon. A stent prior to delivery is disposed about the distal portion. The rotatable sheath may also and/or alternatively be constructed of a non-compliant material where as the balloon is a compliant material.

### CROSS-REFERENCE TO RELATED APPLICATIONS

This application is a continuation application of U.S. application Ser. No. 10/863,724, filed Jun. 8, 2004, entitled “BIFURCATED STENT DELIVERY SYSTEM,” the disclosure of which is hereby incorporated by reference herein.

### BACKGROUND

A stent delivery system employing a stent assembly with branches intended for deployment in the adjacent branches of a vessel bifurcation has been proposed to allow placement of a portion of the assembly in both a primary passage, such as an artery, and a secondary passage, such as a side branch artery. Additionally, these stents generally have an opening which allows for unimpeded blood flow into the side branch artery. However, problems are still encountered in orienting the stent relative to the side branch at the bifurcation of the primary and secondary passages. Moreover, such bifurcated assemblies are typically specially manufactured at an increased cost over a more standard stent intended for single vessel deployment.

In delivering a stent to a vessel location, many current devices rely on either passive torque (e.g., pushing the stent forward and allowing the stent that is fixed on the guidewire/balloon to passively rotate itself into place) or creating torque from outside of the patient to properly orient the medical device in the passage. These devices and methods of achieving proper angular orientation have not been shown to be effective in properly placing and positioning the stent.

Thus, a need exists to provide a catheter which is capable of allowing a medical device such as a stent to be easily maneuvered and aligned at a vessel bifurcation or other location, while also adequately protecting the catheter and/or balloon to which the stent is mounted. Various devices and methods described herein address this need by providing a catheter system with a rotatable sheath apparatus which a stent may be mounted on or engaged to. The rotatable assembly is rotatable about the catheter shaft thereby eliminating the need to apply torque to the catheter shaft to align the stent at a vessel bifurcation.

All U.S. patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### SUMMARY

Catheter systems for delivery of multiple stents or stent segments, wherein at least one of the stents is mounted on the catheter with a freely rotating deployment sheath and assembly are described in U.S. patent application Ser. No. 10/375,689, filed Feb. 27, 2003 and U.S. patent application Ser. No. 10/657,472, filed Sep. 8, 2003 both of which are entitled Rotating Balloon Expandable Sheath Bifurcation Delivery; U.S. patent application Ser. No. 10/747,546, filed Dec. 29, 2003 and entitled Rotating Balloon Expandable Sheath Bifurcation Delivery System; U.S. patent application Ser. No. 10/757,646, filed Jan. 13, 2004 and entitled Bifurcated Stent Delivery System; and U.S. patent application Ser. No. 10/784,337, filed Feb. 23, 2004 and entitled Apparatus and Method for Crimping a Stent Assembly; the entire content of each being incorporated herein by reference.

As used herein the term “stent” refers to an expandable prosthesis for implantation into a body lumen or vessel and includes devices such as stents, grafts, stent-grafts, vena cava filters, etc. In some embodiments a stent may be at least partially constructed of any of a variety of materials such as stainless steel, nickel, titanium, nitinol, platinum, gold, chrome, cobalt, as well as any other metals and their combinations or alloys. A stent may be at least partially constructed of a polymer material. A stent may be at least partially constructed of a shape-memory polymer or material. A stent may be balloon expandable, self-expandable, hybrid expandable or a combination thereof. In some embodiments a stent may include one or more areas, bands, coatings, members etc that is (are) detectable by imaging modalities such as X-Ray, MRI or ultrasound. In some embodiments at least a portion of the stent is at least partially radiopaque. In some embodiments a stent may include one or more therapeutic and/or lubricious coatings applied thereto.

Some embodiments of the present invention are directed to such catheter systems and rotating assemblies wherein the catheter is a balloon catheter having a balloon at least partially constructed of a compliant material and at least one rotatable sheath or sheath section at least partially disposed thereabout which is at least partially constructed of a non-compliant material and/or composite material.

At least one stent is disposed about the at least one sheath or sheath section prior to delivery. A guidewire is moveably engaged to the rotatable sheath and/or stent in order to allow the rotatable sheath to rotatingly align the stent or stents at a vessel bifurcation. In some embodiments the guidewire extends between the stent and sheath exiting radially from a guidewire hole in the wall of the sheath and/or a secondary opening in the stent.

In at least one embodiment the catheter system employs a guidewire housing through which the guidewire is passed. The guidewire housing is fixedly engaged to the rotatable sheath and the stent is disposed thereabout. In some embodiments the guidewire housing extends through the secondary opening of the stent whereupon the guidewire exits the guidewire housing. In some embodiments the guidewire extends from a region of the rotatable sheath proximal to the stent to a distal region and/or distal end of the stent.

In at least one embodiment the guidewire housing has a length of which a majority of is engaged to the rotatable sheath. In some embodiments the entire length of the guidewire housing is engaged to the rotatable sheath. The guidewire housing may be integral with the rotatable sheath, be chemically or adhesively bonded to the rotatable sheath, fused, welded or otherwise engaged to the rotatable sheath.

In at least one embodiment the guidewire housing is constructed at least partially of one or more flexible materials such as Polyisobutylene, Polyurethane, silicone rubber; other synthetic rubbers such as SBS (Styrene Butadiene), SEBS and SIS, latex, etc. In some embodiments at least a portion of the guidewire housing is constructed of a hypotube of nitinol or other metal or alloy which defines one or more substantially spiral shaped cuts or grooves therethrough.

In at least one embodiment a rotatable sheath extends over at least a portion of the balloon and at least a portion of the catheter shaft proximally adjacent thereto. In some embodiments the rotatable sheath has a plurality of longitudinal sections. For example, in at least one embodiment a rotatable sheath has three sections. A first section of a first flexural modulus value, a second section of a second flexural modulus value and a third section of a third flexural modulus value. The first or distal most section is positioned substantially about the balloon and may have a length approximately the same as that of the balloon. The second section is proximally adjacent the first section and the third section is proximally adjacent the second section. The second section and/or third section has/have a different flexural modulus value than that of the first section. In some embodiments the second flexural modulus value is greater than that of the first flexural modulus value but less than the third flexural modulus

In at least one embodiment the rotatable sheath is has a uniform material construction but is provided with sections of differing stiffness and/or flexural modulus by having the wall of the sheath be of varied thickness: providing one or more section of wall with a braided structure, while providing others with different braid or non-braided configurations; providing sections with one or multi-layer construction, pre-stretching one or more layers; selectively ablating or otherwise removing material from one or more layers; etc.

In at least one embodiment for example, a first section of the sheath proximally adjacent to the balloon may have a wall thickness greater than that of a second section of the sheath disposed about the balloon. In some embodiments a region of the sheath wall between the first and second sections may have a tapered thickness.

In at least one embodiment a guidewire underlies at least a portion of the at least one sheath. In some embodiments the guidewire passes through a guidewire opening defined by the wall of the at least one sheath.

In at least one embodiment a first sheath is rotatably disposed about a proximal or first section of the balloon and a second sheath is disposed about a distal or second section of the balloon. The second sheath may be rotatable or non-rotatable about the balloon. In some embodiments a first stent is disposed about the first sheath and a second stent is disposed about the second sheath prior to delivery of the stents. In some embodiments the first sheath and the second sheath at least partially overlap one another. In some embodiments the first sheath and the second sheath are longitudinally spaced apart from one another and define a gap or space therebetween. In some embodiments both the first sheath and the second sheath are at least partially constructed of a non-compliant material. In some embodiments the first sheath has a greater diameter than the second sheath. In some embodiments the first sheath is more compliant than the second sheath.

In at least one embodiment a first sheath is disposed about the balloon. The first sheath having a length at least as great as that of the balloon. A second sheath is rotatably disposed about a distal portion of the first sheath. In some embodiments the distal portion of the first sheath is more or less compliant than the remaining portion(s) of the first sheath. In some embodiments the distal portion of the first sheath defines a plurality of openings or slits wherein the respective areas of the wall of the first sheath have been cut, removed or thinned.

In at least one embodiment a non-compliant sheath is rotatably disposed about the relatively compliant balloon of the catheter. The sheath is provided with a less compliant region in the sheath wall or the sheath is provided a region of the wall having an aneurysm shape. When the non-compliant balloon is expanded the less compliant or aneurysm shaped region of the relatively non-compliant sheath will be pushed or shaped in a radially outward direction to a greater extent than the rest of sheath. In some embodiments a stent having a secondary branch opening defined by a plurality of extension members or fingers is disposed about the sheath, such that when the balloon is expanded the less compliant or aneurysm shaped region of the relatively non-compliant sheath pushes the fingers outward into a branch of a vessel bifurcation.

These and other embodiments which characterize the invention are pointed out with particularity in the claims annexed hereto and forming a part hereof. However, for a better understanding of the invention, its advantages and objectives obtained by its use, reference should be made to the drawings which form a further part hereof and the accompanying descriptive matter, in which there is illustrated and described a embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a side view of a rotating sheath assembly.

FIG. 2 is a side view of the assembly shown in FIG. 1 shown configured for delivery of a stent.

FIG. 3 is a side view of a stent delivery system. The stent delivery system is provided with a rotating collar.

FIG. 4 is a side view of the stent delivery system of FIG. 3 with the rotating sheath assembly and stent of FIG. 2 mounted thereon.

FIG. 5 is a side view of the stent delivery system of FIG. 4 shown being advanced along a guidewire to a vessel bifurcation prior to delivery of the stent.

FIG. 6 is a side perspective view of a stent, such as that shown in FIG. 2.

FIG. 7 is a side perspective view of the stent shown in FIG. 6 wherein a side branch opening is shown formed.

FIG. 8 is a cross-sectional view of the stent of FIG. 7.

FIG. 9 is a side view of the stent depicted in FIG. 5, wherein the stent has been delivered from the stent delivery system, by balloon expansion and the assembly subsequently withdrawn from the vessel(s).

FIG. 10 is a side view of an embodiment of the invention wherein the stent delivery system is provided with a rotatable sheath having differing characteristics along at least part of its length.

FIG. 11 is a side view of an embodiment of the invention wherein the stent delivery system is provided with a rotatable sheath wherein a portion of the sheath wall has a stepped thickness.

FIG. 12 is a side view of an embodiment of the invention wherein the stent delivery system is provided with a rotatable sheath wherein a portion of the sheath wall has a tapered thickness.

FIG. 13 is a cross-sectional view of an embodiment of the invention wherein the stent delivery system is provided with a secondary guidewire housing that is engaged to at least a portion of the rotatable sheath.

FIG. 14 is a cross-sectional view of an embodiment of the invention wherein the stent delivery system is provided with a secondary guidewire housing that is integral with the wall of the rotatable sheath.

FIG. 15A is a perspective view of an embodiment of the invention wherein the balloon and the rotatable sheath of the stent delivery system are shown in the un-expanded state.

FIG. 15B is a perspective view of the embodiment shown in FIG. 15A in the expanded state.

FIG. 16 is a cross-sectional view of the embodiment shown in FIG. 15A.

FIG. 17 is a perspective view of an embodiment of the invention wherein the stent delivery system is shown prior to delivery and is provided with a proximal rotatable sheath and a distal sheath.

FIG. 18 is a perspective view of the embodiment shown in FIG. 17 wherein the balloon is shown in the expanded state during delivery of the stent(s).

FIG. 19 is a perspective view of an embodiment of the invention wherein the stent is shown prior to delivery and the proximal sheath and the distal sheath are configured to partially overlap.

FIG. 20 is a perspective view of the embodiment shown in FIG. 19, wherein the stent is shown in the expanded state.

FIG. 21 is a side view of a first configuration of the sheaths shown in FIG. 19.

FIG. 22 is a side view of a second configuration of the sheaths shown in FIG. 19.

FIG. 23 is a perspective view of an embodiment of the invention wherein the stent delivery system is shown prior to delivery and has a first sheath disposed about the balloon and a proximal rotatable second sheath disposed about the first sheath.

FIG. 24 is a perspective view of an embodiment of the invention illustrated in FIG. 23 shown during delivery of a stent(s).

FIG. 25 is a perspective view of an embodiment of the invention wherein the system is shown configured to expand a crown region of a stent, by pushing the sheath radially outward during balloon expansion to deploy the fingers of the crown region into a side branch of a vessel bifurcation.

FIG. 26 is a partial perspective view of the embodiments shown in FIG. 25 wherein the crown region is depicted prior to delivery.

### DETAILED DESCRIPTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

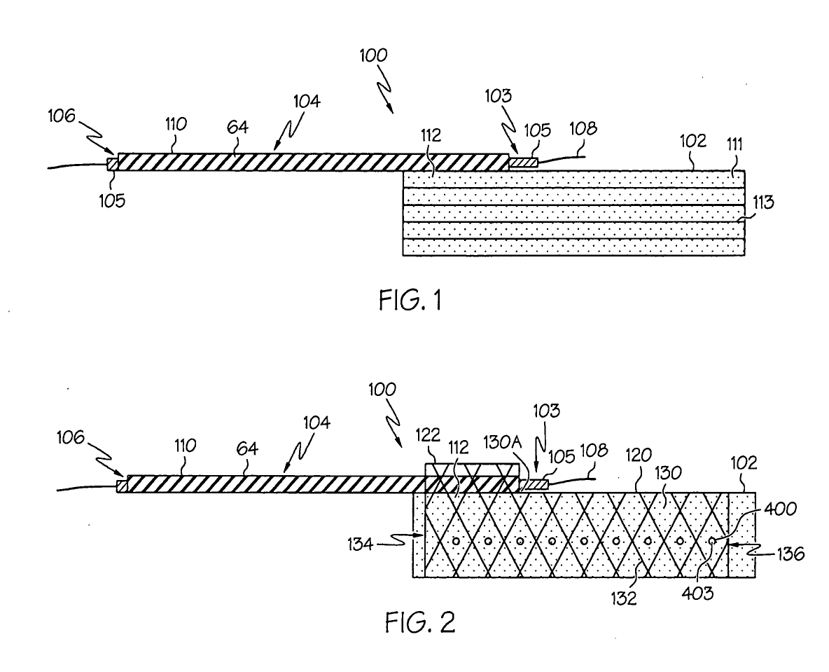

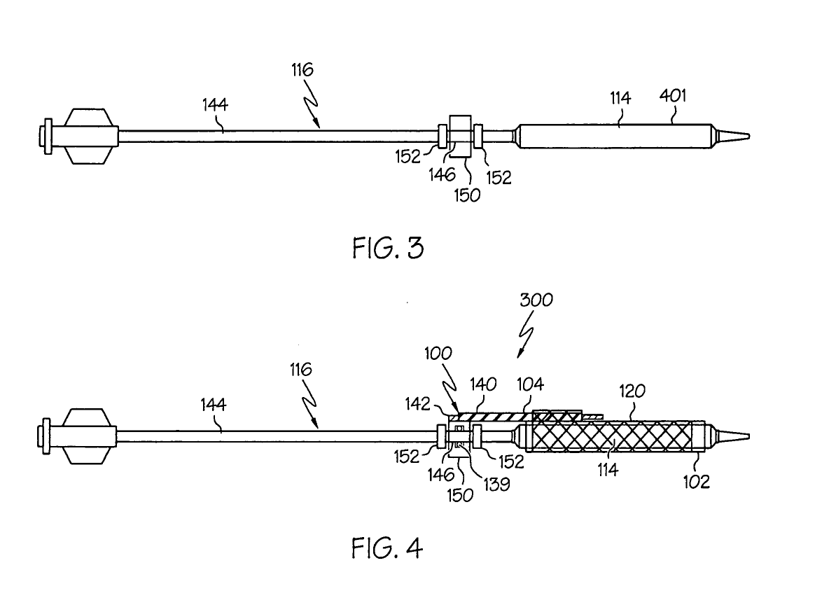

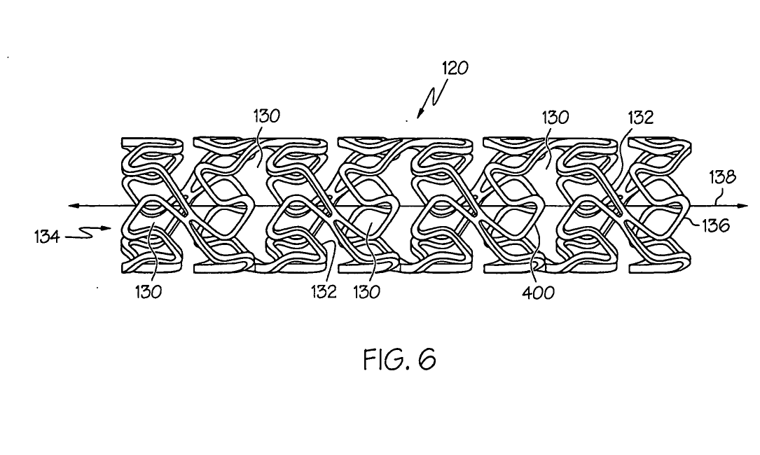

Referring now to the drawings which are for the purposes of illustrating embodiments of the invention only and not for purposes of limiting same, FIGS. 1-2 illustrate a an assembly 100 for use in a stent delivery system 300 which is mounted on a catheter body 116, such as is depicted in FIGS. 3-5, to provide the system with a rotating region that allows a stent 120, such as is shown in FIGS. 6-9, to be properly aligned in a vessel bifurcation. Some additional examples of such assemblies are shown and described in U.S. patent application Ser. No. 10/375,689, filed Feb. 27, 2003 and U.S. patent application Ser. No. 10/657,472, filed Sep. 8, 2003 both of which are entitled Rotating Balloon Expandable Sheath Bifurcation Delivery; U.S. patent application Ser. No. 10/747,546, filed Dec. 29, 2003 and entitled Rotating Balloon Expandable Sheath Bifurcation Delivery System; and U.S. patent application Ser. No. 10/757,646, filed Jan. 13, 2004 and entitled Bifurcated Stent Delivery System, the entire content of each being incorporated herein by reference.

The rotating sheath assembly 100 depicted in FIGS. 1-2 comprises a tubular sleeve or sheath 102 and a positioning or secondary guidewire housing 104. The housing 104 defines a secondary guidewire lumen 106 through which a secondary guidewire 108 may be passed.

Though the housing 104 may be constructed of a wide variety of materials including metal plastic, etc., in some instances the housing 104 may be an external reinforcing member or hypotube 64.

The hypotube 64 may comprise stainless steel, nitinol, one or more polymer materials or other material. To improve flexibility, in some cases the housing 104 is provided with one or more openings 110 along its length. For example, the housing 104 may be spiral cut to provide at least a continuous opening 110 which acts to provide improve the flexibility of the housing 104.

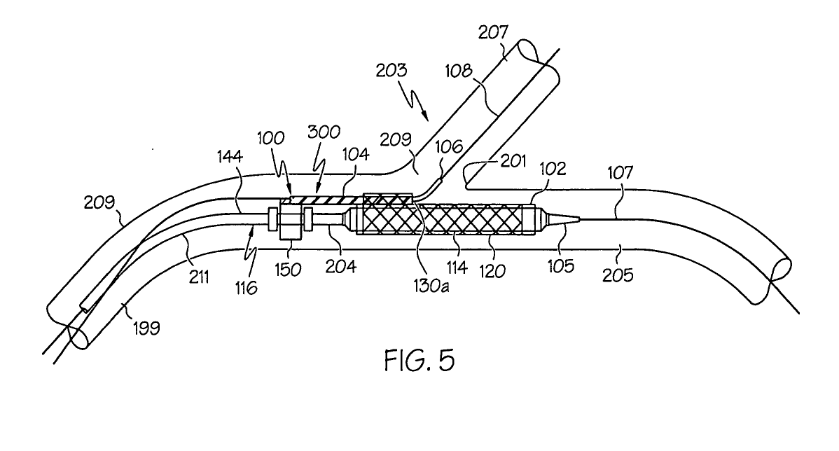

The assembly 100 may include a secondary guidewire housing 104 which further comprises an inner shaft 103, about which the hypotube 64 is disposed. The inner shaft 103 may be a flexible hollow tubular member which extends distally beyond the distal end of the hypotube 64. This distal and/or proximal tips 105 of the inner shaft 103 provides the housing with a flexible protective sheath about the guidewire 108 as it passes out of the secondary guidewire lumen 106. Such a protective covering prevents the guidewire 108 from excessively rubbing against the wall 201 of the vessel 199, such as in the manner depicted in FIG. 5; even where the secondary guidewire 108 exits the secondary lumen 106 at a significant angle. The inner shaft 103 may be constructed of any of a variety of flexible materials such as: PEBAX, nylon, urethane, and/or other materials in a single layer, multi-layer and/or braided configuration.

In many catheters, the shaft 144 of the catheter 116 defines a primary guidewire housing 211 through which a primary guidewire 107 may be advanced. In use, guidewires 107 and 108 are passed through a lumen or other body vessel 199 to a bifurcation 203. Primary guidewire 107 is then advanced into a primary branch of passage 205 of the bifurcation 203 while the secondary guidewire 108 is advanced into the adjacent or secondary branch 207 of the bifurcation 203. As the system is advanced along both guidewires 107 and 108, as a result of the divergent paths defined by the guidewires 107 and 108, the rotatable sleeve 104 will rotate the stent 120 into a desired position so that the secondary opening 130a of the stent is aligned with the secondary passage 207. Where the catheter 116 is a fixed wire system, the use of the primary guidewire is unnecessary.

Examples of the rotating assembly 100 include a distal portion of the housing 104 being engaged to at least a proximal portion of the sheath 102 at an engagement site 112. The manner or mechanism of engagement between the sheath and housing 104 may be by bonding, welding, adhering adhesively engaging, mechanically engaging or otherwise connecting the surfaces of the respective sheath 102 and housing 104.

The sheath 102 is a hollow tube of sheath material that is configured to be placed over the balloon 114 or other region of a catheter 116, such as in the manner illustrated in FIGS. 3 and 4. The sheath 102 is further configured to be rotatable about the catheter shaft and/or balloon 114, even when a stent 120 has been positioned about and/or affixed to the sheath 102.

In order to ensure that the sheath 102 is rotatable about a balloon 114 and/or other region of a catheter, even with a stent 120 crimped on to the sheath 102 and the catheter is being advanced through the a body, the sheath 102 may be constructed of a variety of low friction materials such as PTFE, HDPE, etc. In at least one embodiment the sheath 102 is at least partially constructed of a hydrophilic material, such as hydrophilic polymers such as; TECOPHLIC® material available from Thermedics Polymer Products, a division of VIASYS Healthcare of Wilmington, Mass.; TECOTHANE®, also available from Thermedics Polymer Products; hydrophilic polyurethanes, and/or aliphatic, polyether-based thermoplastic hydrophilic polyurethane; and any other material that provides the sheath 102 with the ability to rotate freely about the balloon 114 when in the “wet” state, such as when the catheter is exposed to body fluids during advancement through a vessel. Suitable sheath materials may also provide the sheath with rotatability in the “dry”, or pre-insertion, state, but with the application of a greater amount of force than when in the wet state, such materials are referred to herein as being tecophilic.

A sheath 102 at least partially constructed from tecophilic material provides the sheath 102 with the ability to rotate freely about the balloon 114 when in the “wet” state, such as when the catheter is exposed to body fluids during advancement through a vessel. The tecophilic sheath 102 is also capable of rotation in the “dry”, or pre-insertion, state, but with the application of a greater amount of force than when in the wet state.

In some cases the sheath 102 may be constructed of one or multiple materials, in one or more layers. For example, the sheath 102 may comprise an outer layer of a softer material than that of the material used in constructing an inner layer, such as has been previously described. In some embodiments, an example of which is shown in FIG. 1, the sheath 102 may be comprised of a matrix of a first material 111 and have one or more supportive stripes, strands, members or areas of a second supportive material 113 within, external to or internal to such a matrix.

The composition of the sheath 102 material, whether a single, multiple layer or stripe reinforced extrusion may include essentially any appropriate polymer or other suitable materials. Some example of suitable polymers include Hydrophilic Polyurethanes, Aromatic Polyurethanes, Polycarbonate base Aliphatic Polyurethanes, Engineering polyurethane, Elastomeric polyamides, block polyamide/ethers, polyether block amide (PEBA, for example available under the trade name PEBAX), and Silicones, Polyether-ester (for example a polyether-ester elastomer such as Arnitel available from DSM Engineering Plastics), Polyester (for example a polyester elastomer such as Hytrel available from Du Pont), or linear low density polyethylene (for example Rexell).

Example of suitable reinforcing materials whether alone or blended with other materials, mixtures or combination or copolymers include all Polyamides (for example, Durethan available from Bayer or Cristamid available from ELF Atochem), polyethylene (PE). Marlex high-density polyethylene, polyetheretherketone (PEEK), polyimide (PI), and polyetherimide (PEI), liquid crystal polymers (LCP), and Acetal (Delrin or Celcon).

Often the inner surface of the sheath 102 or the outer surface of the balloon 114 may include a coating of one or more low friction materials or include one or more low friction materials in its construction. Such a coating 401 is shown in FIG. 3 on the surface of the balloon 114 before assembly 100 has been placed thereabout, such as is depicted in FIG. 4. Coating 401 may however be placed between the balloon 114 and sheath 102 at any time. Some examples of a suitable coating material include but are not limited to: hydrogel, silicon, and/or BIOSLIDE® available from SciMed Life Systems, Inc. of Maple Grove Minn.

As mentioned above, the sheath 102 is configured to be freely rotatable about a balloon of a catheter even when a stent 120, such as is shown in FIGS. 2 and 4 is crimped onto the sheath 102. When properly positioned on the sheath 102, a proximal portion 122 of the stent 120 is also disposed about at least a portion of the secondary guidewire housing 104. When properly positioned about the sheath 102 and the housing 104, at least a portion of the housing 104 and/or the secondary guidewire 108 extends distally through a cell opening 130 of the stent 120.

Stent 120 may be a stent, such as is shown in FIG. 6, which is at least partially constructed of a plurality of interconnected struts, connectors or members 132. The stent 132 defines a proximal opening 134, a distal opening 136 and a flow path 138 therebetween. The cell openings 130 are in fluid communication with the flow path 138.

When the secondary guidewire 108 and/or the secondary guidewire housing 104 is threaded through one of the cell openings 130 when the stent is positioned onto the assembly 100, such as is shown in FIGS. 2 and 4, the members 132 that define the selected cell opening 130a, as well as the shape of the opening 130a through which the secondary guidewire 108 exits the stent, may be distorted or modified in order to accommodate the passage of secondary guidewire 108 and/or the secondary guidewire housing 104 therethrough.

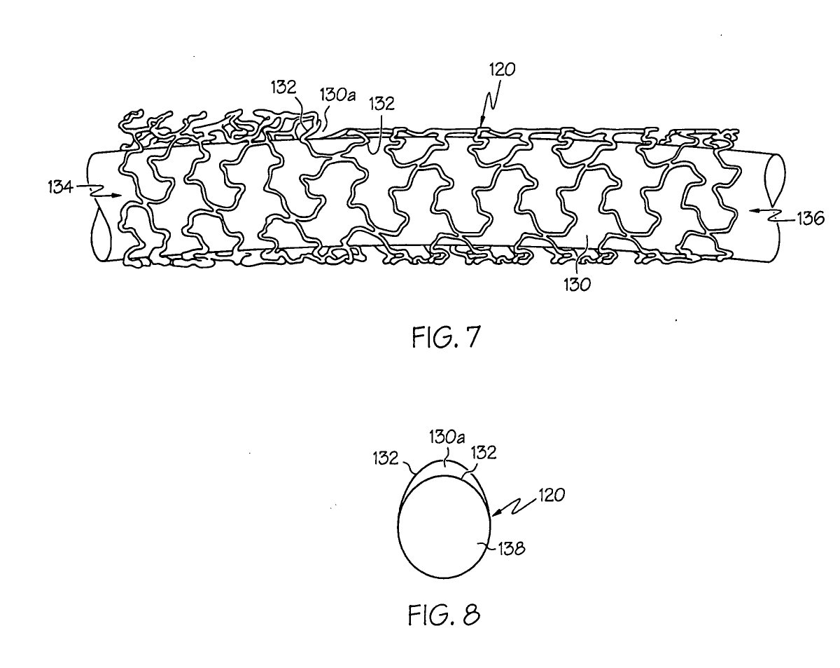

The modified cell opening 130a, hereinafter referred to as secondary opening 130a, is positioned on the stent 120 between the proximal opening 134 and the distal opening 136. The manner in which the secondary opening 130a, the members 132 adjacent thereto, and to an extent the stent 120 itself, are modified or distorted by the position of the secondary guidewire and/or secondary guidewire housing is depicted in FIGS. 7 and 8.

It should be noted that when the stent 120 is placed on the assembly in the manner described above, the distortion of the secondary opening 130a and the adjacent members 132 is of a minimal extent, and is provide only to allow sliding passage of the secondary guidewire 108, and if desired a distal portion of the secondary guidewire housing 104, through the secondary opening 130a. As such, the actual size of the secondary opening 130a may be substantially similar, or only marginally different than that of the surrounding cell openings 130.

It should also be further noted that while stent 120 may be a standard “single vessel” stent that is provided with a secondary opening 130a in the manner described above, the stent 120 may also be a bifurcated stent having a trunk or stem portion, with one or more leg portions and/or branch openings adjacent thereto, through one of which the secondary guidewire may be passed. Such bifurcated stents and stent assemblies are well known in the art.

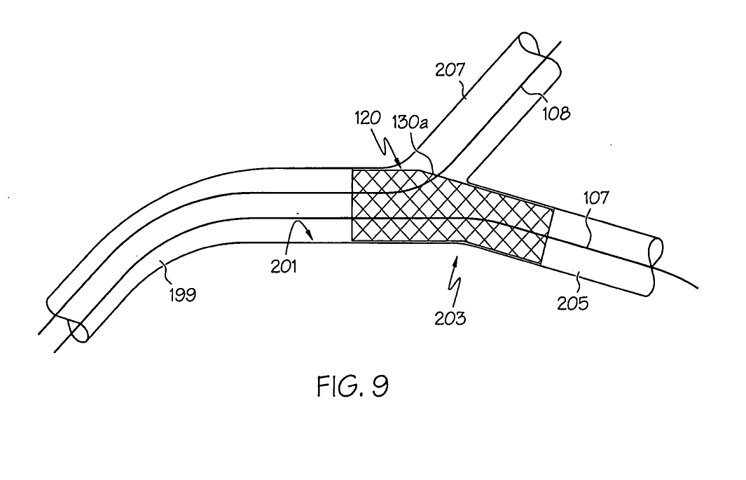

In some cases, the stent 120, sheath 102 or one or more portions thereof, may be configured to deliver one or more therapeutic agents to a delivery site such as within the vessel 199 or one or more areas adjacent thereto, such as shown in FIGS. 5 and 9.

To better accommodate placement of a therapeutic agent on the stent 120, in some instances one or more stent members 132, such as is shown in FIG. 6, maybe configured to include one or more holes, notches, or other surface features to which one or more therapeutic agents 400 may be placed for delivery to the aneurysm site. A therapeutic agent may be placed on the stent in the form of a coating. Often the coating includes at least one therapeutic agent and at least one polymer.

In at least one embodiment, an example of which is shown in FIG. 2, the sheath 102 may include one or more holes, notches, pores, cavities or other surface features 403 wherein one or more therapeutic agents 400 may be positioned. During expansion of the stent 120 the corresponding expansion of the sheath 102 may squeeze or otherwise act to release the agent 400 onto the stent and/or body.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic includes cellular material, the cellular material may include but is not limited to: cells of human origin and/or non-human origin as well as their respective components and/or derivatives thereof. Where the therapeutic agent includes a polymer agent, the agent may be a polystyrene-polyisobutylene-polystyrene triblock copolymer (SIBS), polyethylene oxide, silicone rubber and/or any other suitable substrate.

Once the stent 120 is positioned on the assembly 100, such as in the manner shown in FIG. 2, the assembly 100 may be slid onto a catheter 116, such as is shown in FIGS. 3-4 so that the sheath 102 is rotatingly disposed about the balloon 114 and a proximal portion 140 of the secondary guidewire housing 104 may be engaged to an optional rotating collar 150. The use of collar 150 provides additional securement of the housing 104 to the catheter 116 as well as to minimize longitudinal displacement of the assembly relative to the balloon 114 in the manner described below.

The collar 150 is engaged to the proximal portion 140 of the secondary guidewire housing 104 by any engagement mechanism desired, such as welding, bonding, mechanical engagement, adhesive engagement, etc. As shown in FIG. 4 for example, the proximal portion 140 of the secondary guidewire housing 104 and the collar 150 are engaged externally at engagement site 142. Alternatively, the secondary guidewire housing 104 may be passed at least partially through the collar 150, and/or the collar 150 may define a lumen through which the secondary guidewire 108 may be passed before entering into the secondary guidewire housing 104.

Collar 150 may be a substantially cylindrical member that is disposed about the shaft 144 of the catheter 116 at a position proximal of the balloon 114. The collar 150 may be characterized as defining a catheter shaft lumen 146 through which the catheter shaft 144 is passed. In order to provide the collar 150 with the ability to freely rotate about the catheter shaft 144, the collar 150 defines a catheter shaft lumen 146 which has a diameter greater than the outer diameter of the shaft 144. In some embodiments one or more lubricious substances may be placed between the collar 150 and the shaft 144 to further encourage free rotation therebetween.

While the rotating collar 150 is free to rotate about the shaft 144, in some embodiments it will also be capable of being longitudinally displaced along the shaft 144 as well. As such, in some embodiments one or more locks or hubs 152 may be affixed about the shaft 144 on one or both sides of the collar 150 to prevent or limit the potential longitudinal displacement of the collar 150 relative to the shaft 144. In some embodiments the use of hubs 152 may be avoided or supplemented by providing the catheter shaft 144 with an annular protrusion or ring 139 which the collar 150 may be disposed about to prevent the assembly 100 from experiencing substantial longitudinal migration.

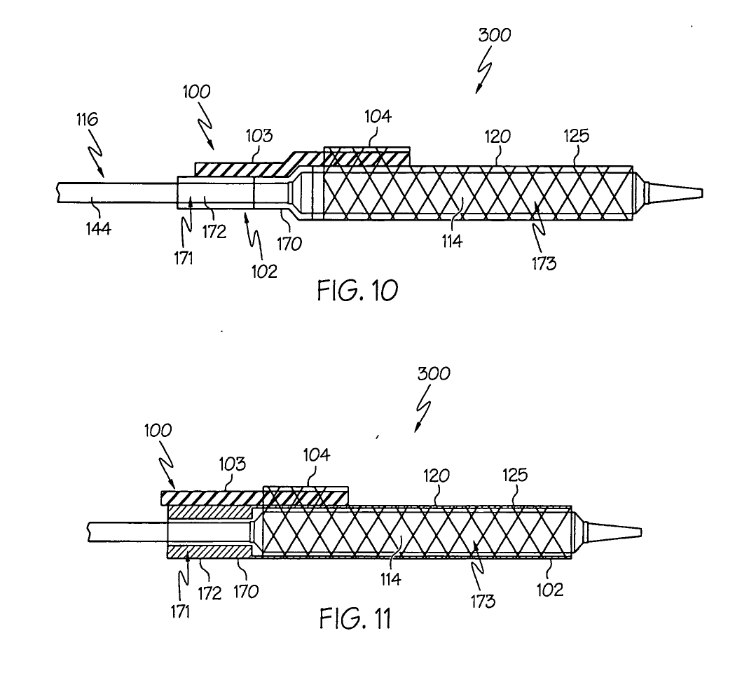

In at least one embodiment, an example of which is shown in FIG. 10, the sheath 102 may be configured to extend proximally beyond the proximal end of the balloon 114 and along a predetermined length of the catheter shaft 144. The length of the sheath 102 while less than that of the length of the catheter shaft 144 may otherwise be of any length desired.

In order to maintain flexibility and trackability of the catheter 116 the sheath 102 may be constructed to include a proximal region 171 that is less flexible, stiffer, and or harder than that of the distal region 173.

In the embodiment shown in FIG. 10 the distal region 173 of the rotatable sheath 102 is disposed about the balloon 114. In at least one embodiment the distal region 173 is at least partially constructed of a material having a lower flexural modulus value than that of the proximal region 171.

In some embodiments the distal region 173 has a flexural modulus value higher than that of the proximal region 171.

Where the proximal region 171 is stiffer than the distal region 173, the proximal region 171 will typically be constructed of material or materials having flexural modulus value(s) of about 300 MPa or more, where as the distal region 173 is constructed of a material or materials having a flexural modulus of about 300 MPa or less. As indicated the regions 173 and 171 may be made stiffer or less stiff as desired, and may likewise be constructed of materials having any of a variety of flexural modulus values.

In some embodiments the proximal region 171 may have multiple sections having different flexural modulus values. For example, in the embodiment shown in FIG. 10 a transition section 170 has a flexural modulus value greater than that of the distal region 173 but less than that of a proximal section 172.

In at least one embodiment the transition section 170 of the sheath 102 defines a portion of the sheath 102 wherein at least the inner diameter of the sheath necks down or transitions from the greater diameter about the balloon to a lesser diameter about the catheter shaft 144. Though such necking down of the sheathes' inner diameter is not necessary, the transition does provide the sheath 102 with a bias relative to the proximal end of the balloon 114 which may aid in preventing longitudinal displacement of the sheath 102 during advancement of the system 300.

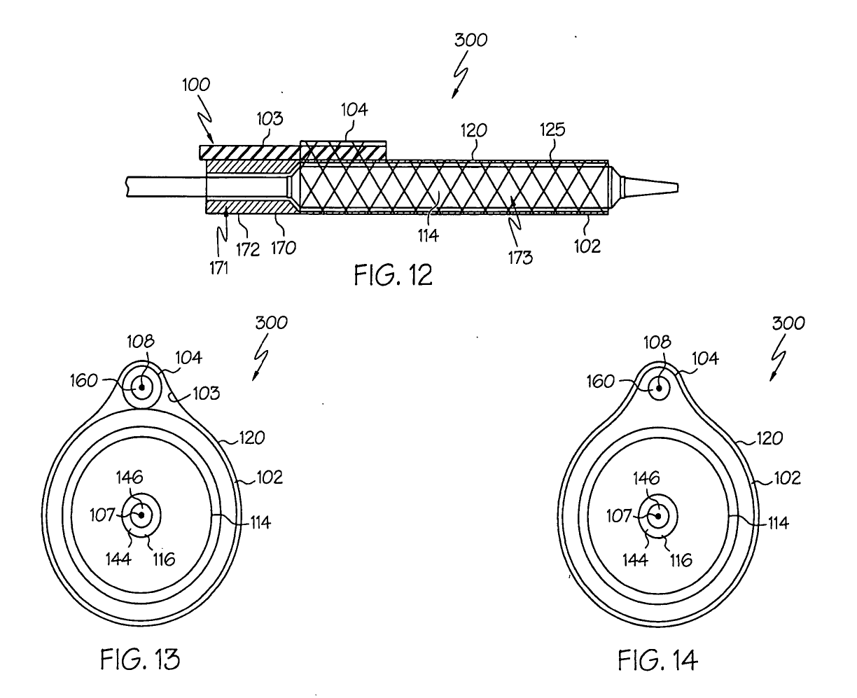

In some embodiments, such as that shown in FIGS. 11 and 12, the sheath 102 may be provided with different regions of stiffness by providing a sheath 102 of a continuous material construction but which has a thinner wall thickness in the distal region 173 than in the proximal region 171. A transition section 170 may be provided where the inner diameter of the sheath 102 is stepped, as in the case of the embodiment shown in FIG. 11, or tapered, as in the case of the embodiment shown in FIG. 12 between the region of the sheath which is disposed about the balloon 114 and the catheter shaft 144.

In some embodiments one or more regions or sections of the sheath 102 may be provided with cuts, slits, indentations or other openings or pores in the wall of the sheath 102 to vary the flexibility and/or stiffness of a respective region or section. Likewise, in some embodiments a coating of a hardening agent or other material(s) may be applied to one or more sections or regions of the sheath 102 in order to modify the hardness, flexibility, and/or stiffness of a respective region or section.

As shown in FIGS. 10-12, the increased length of the sheath 102 provides the assembly 100 with a longer engagement surface is between the sheath 102 and the secondary guidewire housing 104. The secondary guidewire housing 104 may be engaged along a majority or its entire length to the rotatable sheath 104. By providing a more extensive engagement between the housing 104 and sheath 102 the need of a hypotube or other relatively hard outer layer is unnecessary in the construction of the guidewire housing 104 as sufficient stiffness may be provided by at least the proximal region 171 of the sheath 102.

In the embodiments shown in FIGS. 10-12 the housing 104 may be comprised of the relatively flexible inner shaft 103 such as has been described above. The housing 104 may be adhesively or chemically bonded to the sheath 102 and/or may be fused welded or otherwise engaged to the sheath 102 such as in the manner depicted in FIG. 13.

In some embodiments the housing 104 may be integral with the wall of the sheath 102 such as is shown in FIG. 14. In such an embodiment a guidewire opening may be provided radially through the housing 104/sheath 102 in order to allow the secondary guidewire 108 to exit the secondary guidewire lumen 106. In some embodiments the lumen 106 may extend through the length of the sheath 102.

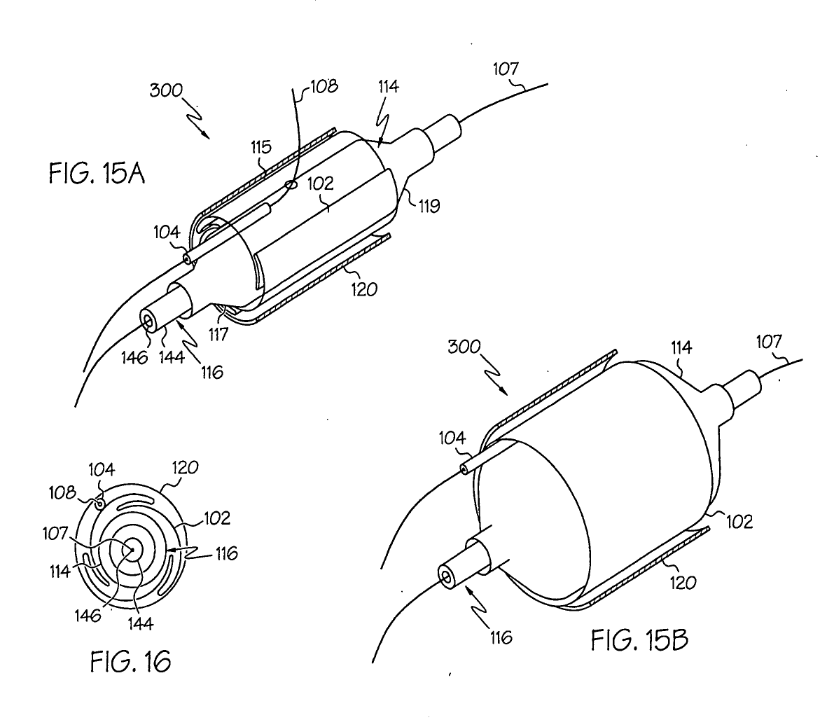

In some embodiments, an example of which is shown in FIG. 15A, the rotatable sheath 102 has a length which is about the same as, or somewhat greater than the length of the balloon body 115. In the embodiments shown the sheath 102 is constructed of one or more non-compliant materials whereas the balloon 114 is constructed of one or more compliant materials.

When the balloon 114 is unexpanded during advancement of the system, the sheath 102 is folded or wrapped around the balloon 114, such as in the manner illustrated in FIGS. 15A and 16. The non-compliant nature of the sheath 102 allows the sheath 102 to be freely rotatable about the balloon when folded thereabout in the folded or “unexpanded” state. When the balloon is expanded, as shown in FIG. 15B, the sheath will unfold or unwrap to its nominal unfolded or “expanded” diameter. The non-compliant nature of the sheath 102 allows the nominal diameter of the sheath 102 to be selected in order to limit or alter the expansion of the more compliant balloon 114. In some embodiments, by providing the sheath 102 with tapered end regions the unfolded sheath 102 is biased against the respective cones 117 and 119 of the balloon 114 thereby ensuring that the sheath 102 cannot be substantially longitudinally displaced relative to the balloon 114.

In loading the catheter 116, the non-compliant sheath 102 is slid over the balloon and placed in the folded reduced diameter condition. Once in place the stent 120 is positioned over the sheath 102 and crimped on top of the sheath 102 as well as the secondary guidewire housing 104 if desired. In some embodiments the housing 104 is engaged to the sheath 102 by adhesive, chemical, mechanical, or other form of engagement prior to mounting the stent 120 about the sheath 102 and housing 104.

In some embodiments the non-compliant sheath 102 is constructed of one or more materials including, but not limited to: Nylon 12, Polyethylene terephthalate (PET), polybutylene terephthalate (PBT), Polyamide 12, Polyether block amide (PEbax) 7233, Pebax 7033, PTFE, Polyaryletherketones (PEEK), Polyphenylene Oxide (PPO), etc. Other materials include the use reinforcing fibers such as HDPE, stainless steel, and others which may be braided and/or covered by any polymer (non-compliant as well as compliant) as the braiding is providing the non-compliant character.

In some embodiments the compliant balloon 114 is constructed of one or more materials including, but not limited to: silicon rubber, urethane, Polyisobutylene, Polyurethane, SBS, SEBS and SIS, etc.

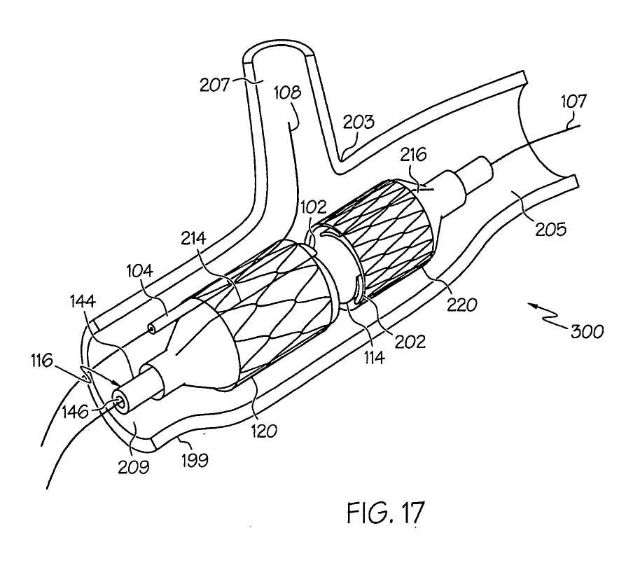

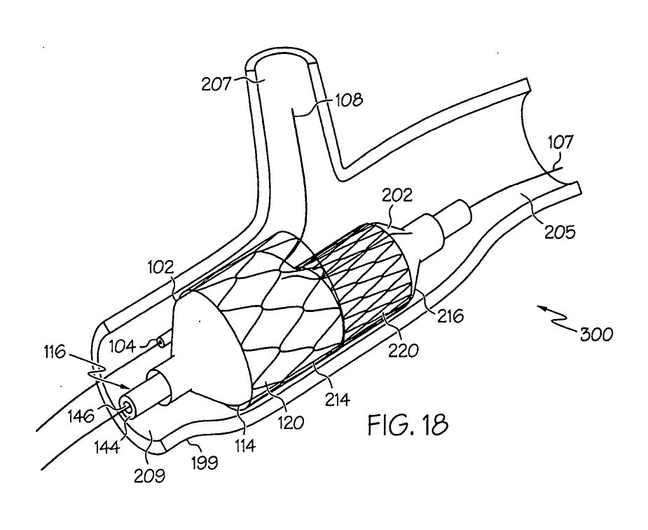

As indicated above the use of non-compliant material or materials in the construction of the rotatable sheath 102 provides the ability to tailor the expansion of the compliant balloon 114. For example, in some embodiments, an example of which is shown in FIG. 17, the system 300 may be configured to deploy two stents 120 and 220 at a vessel bifurcation 203. Because it may be desirable to deploy the first stent 120 into the typically larger diameter main branch 209 of the vessel 199 proximal to the bifurcation 203 and/or at least partially across the opening of a side branch 207, and the second stent 220 into the typically narrower side branch 205 distal of the bifurcation 203, each stent may be disposed about separate non-compliant sheaths 102 and 202. Only one, such as sheath 102, or both sheathes 102 and 202 may be rotatable about the balloon 114. Where only one sheath 102 is rotatable, the other sheath 202 may be engaged by welding, adhesion or other engagement mechanism to the catheter shaft 144 and/or balloon 114.

In order to properly deploy the two stents 120 and 220 to a vessel or vessels having different diameters, the balloon 114 must be capable of expanding each sheath and thus each stent to the appropriate extent. Rather than modifying the construction of the balloon, in some embodiments sheaths 102 and 202 are constructed of a substantially non-compliant material, wherein the sheaths have different nominal diameters when the balloon is expanded. Because of their non-compliant nature, the sheaths will limit expansion of the respective portion of the balloon about which they are disposed to the desired nominal diameter of each sheath. For example, in the embodiment shown in FIGS. 17 and 18, the rotatable proximal sheath 102 has a nominal diameter greater than that of the distal sheath 202. As such, when the balloon is expanded to deliver the stents 120 and 220, such as in the manner shown in FIG. 18, the distal region 216 of the balloon 114 will expand only to the extent permitted by the nominal diameter of the distal sheath 202, and the proximal region 214 of the balloon 114 will expand to a greater diameter limited to the nominal diameter of the proximal sheath 102.

As a result, stent 120 is expanded to a greater deployed diameter than the distally positioned stent 220. If desired expansion of the balloon 114 may be controlled by using sheathes of different construction, multiple sheathes, stent configuration, and/or by modifying the expansion characteristics of the balloon and/or catheter. In some embodiments different stents may be expanded or limited to the same or different diameters and to any extent desired in accordance with the concepts described above.

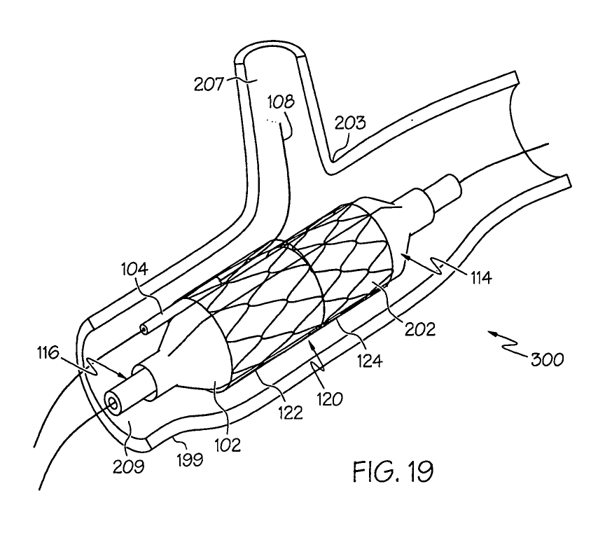

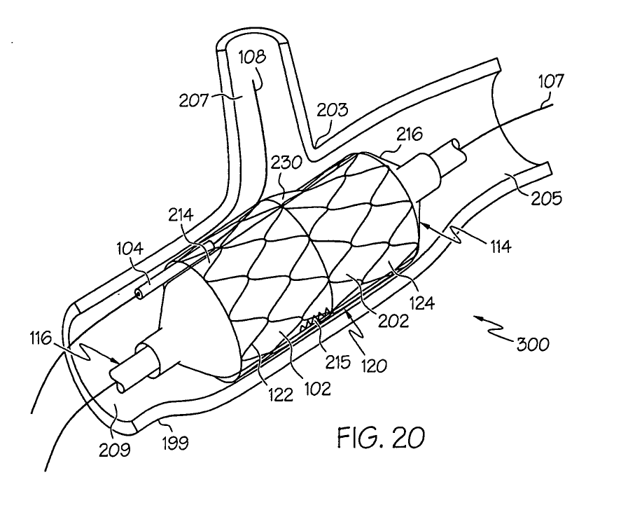

In some embodiments, it may be necessary or desirable to expand a single stent 120 in such a manner that a proximal portion 122 expands to a different diameter than the distal portion 124 such as in the manner shown in FIGS. 19 and 20. In such an instance the stent may be mounted about to rotatable sheathes 102 and 202 of substantially non-compliant construction, wherein one of the sheaths has a nominal diameter greater than that of the other. In the present embodiment, the proximal rotatable sheath 102 has a nominal diameter greater than that of the distal rotatable sheath 202. As a result when the relatively compliant balloon 114 is expanded, the distal region 216 of the balloon, and likewise the distal region 124 of the stent 120, will be limited in expansion by the nominal diameter of the non-compliant distal sheath 202. The proximal region 214 of the balloon 114, and likewise the proximal region 122 of the stent 120, will be expanded to a greater extent than the respective distal regions being limited by the nominal diameter of the substantially non-compliant proximal sheath 102.

As is shown in FIG. 19, at least one of the sheaths 102 and/or 202 may be formed at an angle to provide the sheaths with an overlapping region 215. The sheathes may be independently rotatable prior to delivery or may be engaged to one another at the overlapping region by welding, adhesive engagement, mechanical engagement or other engagement mechanisms. In some embodiments, as a result of the angled configuration of the overlapping sheathes 102 and 202 a region of the sheaths circumferentially adjacent and/or opposite the overlapping region 215 a guidewire gap 230 is defined by the portions of the sheathes that are separated from one another. In some embodiments the presence of the guidewire gap 230 allows the system 300 to be configured with the guidewire housing 104 and/or the guidewire 108 to underlay the proximal sheath 102 and pass radially outward through a secondary stent opening 130a which lies over the gap 230, however as shown in FIGS. 19 and 20 the secondary guidewire housing 104 may be positioned on the exterior of the sheath 102 as shown. In some embodiments the guidewire housing may be integral with the construction of the proximal sheath 102 as previously described. In embodiments wherein the housing 104 is positioned under the proximal sheath 102, the housing is configured so as to not substantially interfere with the rotatability of the sheath 102 about the balloon 114.

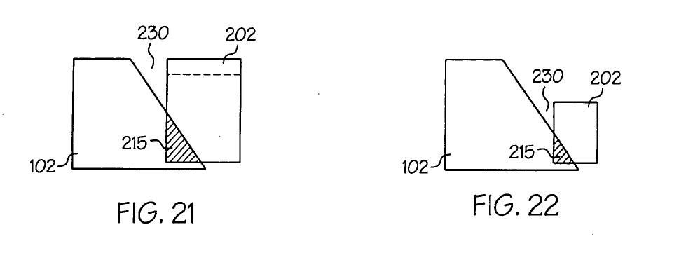

As illustrated in FIGS. 21 and 22 the sheaths 102 and 202 may be configured to overlap to a variety of extents. Also, the nominal diameter of either or both sheathes may be varied.

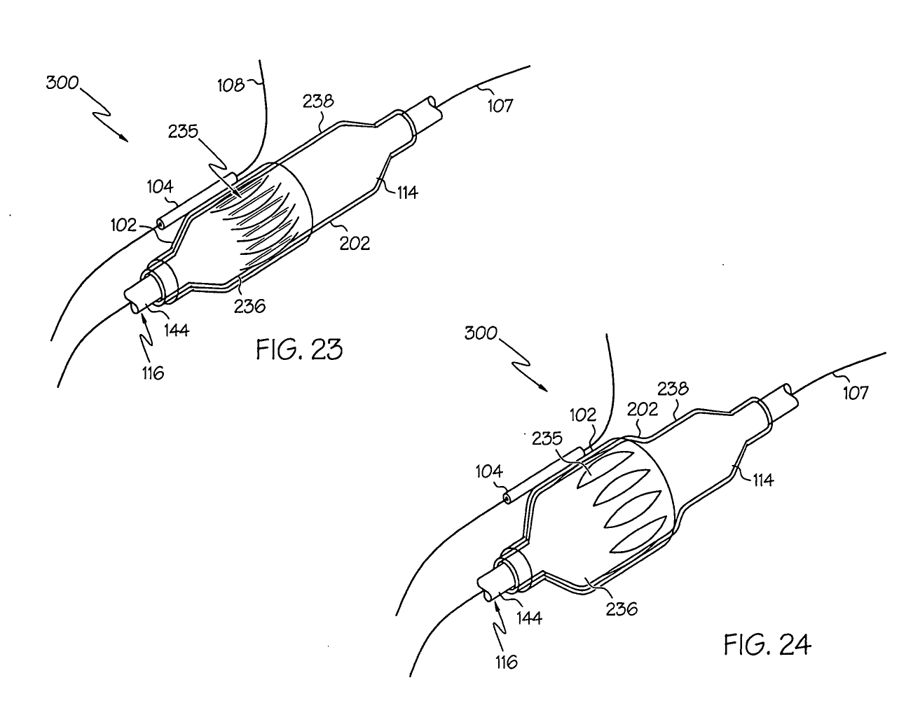

In some embodiments, such as in the example shown in FIGS. 23 and 24, the expansion characteristics of a compliant balloon 114 may be modified by providing the balloon with a cover, sheath or sleeve 202 which has been structurally modified to allow the balloon 114 to expand in one region to a greater extent than in another.

As an initial note, the for illustrative purposes the system 300 depicted in FIGS. 23 and 24 is not shown with a stent or stents thereon. It will be recognized however, that the system 300 shown could of course be utilized with or without a stent or stents as is the case with all of the embodiments of the system 300 described herein.

In the embodiment shown in FIGS. 23 and 24, the balloon cover 202 is a sleeve of substantially non-compliant material which has a length extending over substantially the entire balloon. A region of the cover 202, in this instance the proximal region 236 of the sheath 202, defines a plurality of openings, slits, cuts, pores, thinned areas, etc. 235, through the cover wall. The openings 235 allow the portion of the balloon 114 there under to expand to a greater effective diameter than the portion of the balloon underlying the distal region 238 of the sheath 202 which has no or fewer openings therethrough. As is illustrated in FIGS. 23 and 24, the openings 235 allow the non-compliant sheath to bulge out in the slitted area at the expense of axial shortening.

The balloon cover 202 may be rotatable about or fixedly engaged to the balloon 114 and/or catheter shaft 144 at one or more locations. Rotatably disposed about at least the proximal region 236 of the balloon cover 202 is a rotatable sheath 102 such as has been previously described. In some embodiments a secondary guidewire housing 104 is engaged or is a part of the rotatable sheath 102 such as in any of the manners previously described.

In practice a first stent is mounted about the rotatable sheath 102 and in some embodiments a second stent is disposed about the distal region 238 of the balloon cover 202. As a result of the rotation provided by the rotatable sheath 102 the first stent is independently rotatable about the balloon 114 as the system is advanced through a lumen or vessel. The direction and degree of rotation of the stent and sheath 102 is a consequence of the advancement of the system along the guidewire 108 which has been previously described above in.

Once the system 300 is properly positioned at a vessel bifurcation the compliant balloon 114 is expanded to deliver the stent or stents in the manner previously depicted and described. As the balloon 114 pushes outward against the balloon cover 202, the distal region 238 of the cover 202 will limit the balloons expansion to that of the nominal diameter of the cover 202. The openings 235 in the proximal region 236 of the balloon cover 202 allow the cover 202 to bulge outward in the region of the openings 235 at the expense of axial shortening such as is illustrated in FIGS. 23 and 24. The proximal portion of the balloon may continue expanding until it reaches the limiting nominal diameter of the rotatable sheath 102. As a consequence, stents mounted about the rotatable sheath 102 and/or covering sheath 202 will be expanded to different diameters is indicated by the expansion of the balloon 114 shown in FIG. 24.

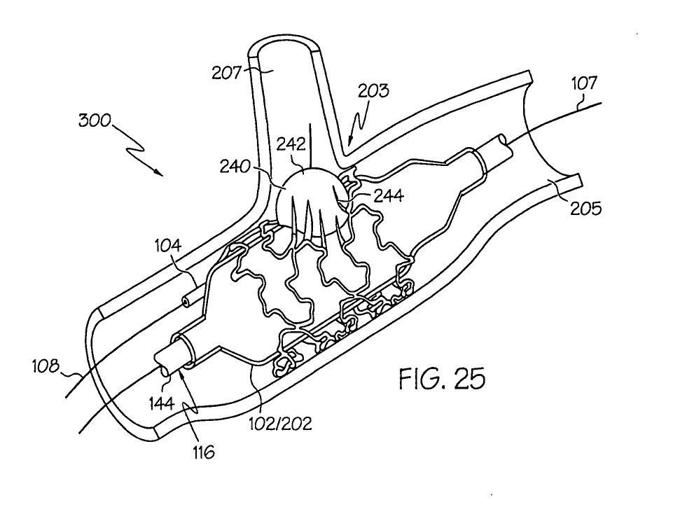

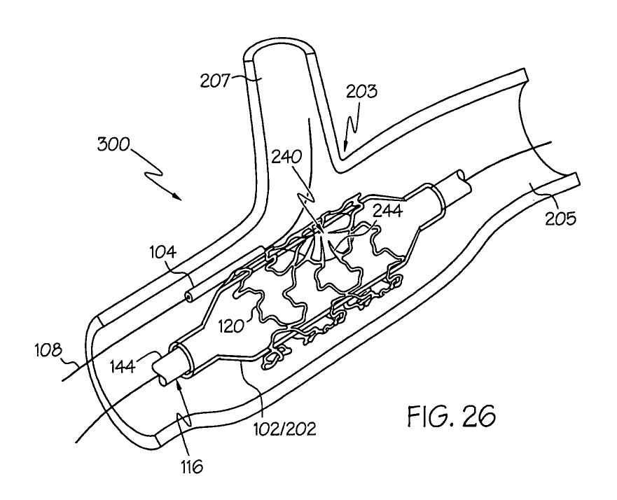

In any of the various embodiments described above, a sheath such as sheath 102 and/or 202 may be provided with an opening, weakened or thinner area, or a predetermined shape which allows the compliant balloon to directly or indirectly deploy a portion of a stent 120, such as a crown region 240 as depicted in FIGS. 25 and 26, into a side branch 207 of a vessel bifurcation 203.

Where the sheath 102 and/or 202 is a non-compliant material the sheath may be provided with a predetermined shape such that in the nominal or expanded diameter a predetermined region or protrusion 242 of the sheath extends radially outward to a greater extent than the rest of the sheath (i.e. protrudes away from the balloon). The protrusion 242 is formed as the expansion of the compliant balloon 114 is directed into the region of the protrusion 242 during balloon inflation. The protrusion 242 will act upon the individual extension members 244 of the crown 240 which otherwise rest substantially within the circumferential plane of the stent as illustrated in FIG. 26, by pushing them radially outward and away from the rest of the stent 120 during expansion. As a result of this pushing action the crown 240 is deployed into the side branch as shown in FIG. 25.

Furthermore, it is noted that the various embodiments shown and described in U.S. patent application Ser. No. 10/375,689, filed Feb. 27, 2003 and U.S. patent application Ser. No. 10/657,472, filed Sep. 8, 2003 both of which are entitled Rotating Balloon Expandable Sheath Bifurcation Delivery; U.S. patent application Ser. No. 10/747,546, filed Dec. 29, 2003 and entitled Rotating Balloon Expandable Sheath Bifurcation Delivery System; U.S. patent application Ser. No. 10/757,646, filed Jan. 13, 2004 and entitled Bifurcated Stent Delivery System; and U.S. patent application Ser. No. 10/784,337, filed Feb. 23, 2004 and entitled Apparatus and Method for Crimping a Stent Assembly may be incorporated and/or utilized with the various embodiments described herein.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

With this description, those skilled in the art may recognize other equivalents to the specific embodiment described herein. Such equivalents are intended to be encompassed by the claims attached hereto.

We claim:

1. A stent delivery system comprising:
a catheter body;
a balloon disposed about the catheter body, the balloon being constructed of at least one substantially compliant material, the balloon being expandable from an unexpanded state to an expanded state;
a first sheath portion rotatably mounted about the balloon to rotate with respect to the balloon, the first sheath portion being expandable from an unexpanded condition to an expanded condition when the balloon is expanded from the unexpanded state to the expanded state, the first sheath portion having a first diameter when arranged in the expanded condition;
a first stent portion configured to expand from an unexpanded configuration to an expanded configuration, the first stent portion being disposed about the first sheath portion when the first stent portion is arranged in the unexpanded configuration;
a second sheath portion rotatably mounted about the balloon to rotate with respect to the balloon, the second sheath portion being axially aligned with the first sheath portion, the second sheath portion being expandable from an unexpanded condition to an expanded condition when the balloon is expanded from the unexpanded state to the expanded state, the second sheath portion having a second diameter when arranged in the expanded condition, the second diameter being less than the first diameter; and
a second stent portion configured to expand from an unexpanded configuration to an expanded configuration, the second stent portion being disposed about the second sheath portion when when the second stent portion is arranged in the unexpanded configuration.

2. The stent delivery system of claim 1, wherein the at least the first sheath portion is folded about the balloon when the first sheath portion is arranged in the unexpanded condition, the first sheath portion being unfolded from about the balloon when the first sheath portion is arranged in the expanded condition.

3. The stent delivery system of claim 2, wherein the second sheath portion is folded about the balloon when the second sheath portion is arranged in the unexpanded condition, the second sheath being unfolded from about the balloon when the second sheath portion is arranged in the expanded condition.

4. The stent delivery system of claim 1, wherein the first sheath portion is constructed from a first material and the second sheath portion is constructed from a second material, the first material being less stiff than the second material.

5. The stent delivery system of claim 4, wherein the first material has a flexural modulus value less than a flexural modulus value of the second material.

6. The stent delivery system of claim 1, wherein the first sheath portion includes a tapered end region.

7. The stent delivery system of claim 6, wherein the tapered end region inhibits substantial longitudinal displacement of the first sheath portion with respect to the balloon.

8. The stent delivery system of claim 1, further comprising:
a secondary guidewire housing coupled to at least the first sheath portion, the secondary guidewire housing defining a guidewire lumen for passage of a secondary guidewire therethrough.

9. The stent delivery system of claim 8, wherein the secondary guidewire housing is integral with the first sheath portion.

10. The stent delivery system of claim 8, wherein the secondary guidewire housing is coupled to an interior of the first sheath portion.

11. The stent delivery system of claim 8, further comprising:
a secondary guidewire configured to extend through the secondary guidewire housing.

12. The stent delivery system of claim 11, wherein a distal portion of the secondary guidewire housing extends in a substantially radial direction through a secondary opening of the first stent portion.

13. The stent delivery system of claim 11, wherein a distal portion of the secondary guidewire housing extends through the first stent portion from a proximal end of the first stent portion to a distal end of the first stent portion.

14. The stent delivery system of claim 1, wherein the first stent portion is physically separate from the second stent portion.

15. The stent delivery system of claim 1, wherein the first sheath portion is physically separate from the second sheath portion.

16. The stent delivery system of claim 15, wherein a section of the first sheath portion overlaps a section of the second sheath portion to form an overlapping section when the first and second sheath portions are disposed about the balloon.

17. The stent delivery system of claim 15, wherein the first sheath portion and the second sheath portion define a gap circumferentially adjacent to or opposite from the overlapping section.

18. The stent delivery system of claim 17, wherein a distal portion of the secondary guidewire housing extends though the gap defined by the first and second sheath portions.

19. The stent delivery system of claim 18, wherein the first sheath portion is constructed from a first material and the second sheath portion is constructed from a second material, the first material being less stiff than the second material.
